(19) United States
(12) Patent Application Publication
(10) Pub. No.: US 2014/0058200 A1
(43) Pub. Date: Feb. 27, 2014

(54) ENDOSCOPIC CAP

(71) Applicant: NinePoint Medical, Inc., Cambridge, MA (US)

(72) Inventors: Shekhar Nimkar, Swampscott, MA (US); James W. Moriarty, JR., Georgetown, MA (US); Narissa Y. Chang, Somerville, MA (US)

(73) Assignee: NinePoint Medical, Inc., Cambridge, MA (US)

(21) Appl. No.: 13/965,575

(22) Filed: Aug. 13, 2013

Related U.S. Application Data
(60) Provisional application No. 61/692,000, filed on Aug. 22, 2012.

(51) Int. Cl. A61B 1/00

(52) U.S. Cl. CPC A61B 1/00154
USPC 600/115; 600/114

(57) ABSTRACT

An endoscope support device is provided. The device includes a sleeve extending along a longitudinal axis between a first end and a second end. The sleeve includes an inner surface defining a passageway configured for disposal of an endoscope. An end surface of the distal end is spaced from external surroundings by a wall that defines the end surface. A center piece is disposed within the passageway. The center piece includes a first end that engages the end wall and an inner surface defining a lumen configured for disposal of an optical probe. The center piece is positioned within the passageway such that the lumen of the center piece is aligned with a lumen of the endoscope and the probe extends through the lumens. Systems, kits and methods are also provided.

### FIELD OF THE INVENTION

This disclosure relates generally to medical devices, and more particularly to a surgical system for imaging tissue within the anatomy of a patient, such as, for example, esophageal tissue. The surgical system includes optical imaging devices used in biomedical and other medical and non-medical applications where imaging using an optical source is utilized. In particular, the surgical system includes an optical probe positioned within an endoscope cap. The endoscope cap aids in centering the optical probe and extends the area available for imaging using the optical probe.

### BACKGROUND OF THE INVENTION

Various types of devices are used in conjunction with an endoscope to aid in centering the probe portion of the endoscope. The available devices use large inflatable members such as, for example, balloons for positioning an optical probe in a selected area of a patient's anatomy. These devices are used during medical procedures to expand and press against an internal cavity of a patient so that an optical probe may be used to image a portion of the internal cavity of a patient such as, for example, the esophagus.

One type of inflatable member is a balloon catheter. In general, balloon catheters can move between a deflated state and an inflated state; intermediate states are also available. In use, the balloon catheter is inserted into the anatomy of a patient while in the deflated state. After selectively positioning the balloon catheter within the patient, the balloon catheter is inflated via any of various means using various inflation media. For example, a syringe may be used to inject liquid into the balloon or an inflation bulb may be used to provide air into the balloon. Some systems utilize a pressure gauge to monitor the pressure of the balloon to prevent over pressurization of the balloon.

To capture clear images of tissue that defines an internal cavity of a patient, an imaging device, such as, for example, an optical probe can be positioned within a balloon catheter before the balloon catheter is inserted into the cavity. The balloon is then inflated to provide access for the imaging device. However, the probe is typically difficult to center since it is often large and pliable, which also makes steadying the probe challenging. Misalignment of the probe can lead to poor quality images. Over or under inflating the balloon may also lead to improperly captured images of the surrounding tissue. This disclosure describes an improvement over these prior art technologies.

### SUMMARY

An endoscope support device is provided in accordance with the principles of the present disclosure. The endoscope support device includes a sleeve extending along a longitudinal axis between a first end and a second end. The sleeve includes an inner surface defining a passageway configured for disposal of an endoscope. An end surface of the distal end is spaced apart from external surroundings by a wall that defines the end surface. A center piece is disposed within the passageway. The center piece includes a first end that engages the end wall and an inner surface defining a lumen configured for disposal of an optical probe. The center piece is positioned within the passageway such that the lumen is aligned with a lumen of the endoscope and the probe extends through the lumens.

In one embodiment, in accordance with the principles of the present disclosure, the endoscope support device described in the preceding paragraph includes an inflatable balloon having a first end attached to an exterior surface of the sleeve at a first point and a second end attached to the exterior surface at a second point. The balloon is attached to an inflating device that directs an inflation material, such as, for example, pressurized gas or fluid from the inflating device to an interior of the balloon to expand the balloon about the sleeve.

In some embodiments, in accordance with the principles of the present disclosure, the endoscope support device includes lay flat tubing designed to be placed along the entire length of the endoscope support device such that the lay flat tubing acts as a protective shield from bodily fluids and damage when an endoscope is inserted into an internal cavity of a patient, such as, for example, the esophagus of the patient. In one embodiment, the lay flat tubing is attached to the sleeve such that the lay flat tubing acts as a tether, by protecting the sleeve from going down the esophagus unintentionally.

In one embodiment, in accordance with the principles of the present disclosure, a method for imaging an internal cavity of a patient's anatomy is provided using the endoscope support device described above in paragraphs [0005] and [0006]. The method includes positioning an end portion of an endoscope into the passageway so that the center piece is coaxial with an optical probe of the endoscope; positioning the endoscope support device into anatomy of a patient; advancing the optical probe through a probe assembly lumen of the endoscope and into the lumen of the center piece; inflating the balloon so that the balloon contacts at least a portion of a wall defining an internal cavity of a patient so as to hold and/or center the device within the internal cavity; and activating the optical probe to obtain and record images of at least a portion of a tissue defining the internal cavity.

In one embodiment, in accordance with the principles of the present disclosure a kit for an endoscope is provided that includes at least one of the endoscope support devices described above in paragraphs [0005] and [0006]. The kit further includes tubing for supplying air from the inflating device to the interior of the balloon, additional replacement tubing attachable to the air supply device and additional balloons attachable to the sleeve. In some embodiments, lay flat tubing is supplied as part of the kit. The kit may also be equipped with different sized support devices that can be used depending on size of the patent and the procedure in which the endoscope is being deployed.

In one embodiment, in accordance with the principles of the present disclosure, an endoscope support device is provided that includes a sleeve extending along a longitudinal axis between a first end and a second end. The sleeve includes an inner surface defining a passageway configured for disposal of an endoscope. An end surface of the distal end is spaced from external surroundings by a wall that defines the end surface. A center piece is disposed within the passageway. The center piece includes a first end that engages the end wall and an inner surface defining a lumen configured for disposal of an optical probe. The center piece is positioned within the passageway such that the lumen is aligned with a lumen of the endoscope and the probe extends through the lumens. An inflatable balloon comprises a first end attached to an exterior surface of the sleeve at a first point and a second end attached to the exterior surface at a second point. A first end of an inflating device is positioned in the passageway. The inflating device includes an inner surface that defines a channel that is communication with an interior of the balloon such that the channel directs fluid from the inflating device to the interior of the balloon to expand the balloon about the sleeve.

In one embodiment, in accordance with the principles of the present disclosure, a surgical system is provided. The surgical system includes an elongated sleeve extending along a longitudinal axis between a first end and a second end. The sleeve includes an inner surface defining a passageway. An end surface of the second end is spaced from external surroundings by a wall that defines the end surface. An elongated center piece is disposed within the passageway. The center piece comprises a first end that engages the end wall and an inner surface defining a lumen configured for disposal of an optical probe. An endoscope is disposed within the passageway adjacent the first end of the sleeve. The endoscope includes an inner surface defining an optical probe lumen that is continuous with the lumen of the center piece. An optical probe is disposed within the optical probe lumen and the lumen of the center piece. At least one of the sleeve and the center piece is constructed of high light transmission and low light scattering material.

### BRIEF DESCRIPTION OF THE DRAWINGS

The present disclosure will become more readily apparent from the specific description accompanied by the following drawings, in which:

FIG. 1 is a side, cross sectional view of a system in accordance with the principles of the present disclosure;

FIG. 2 is a side, cross sectional view of the components of the system shown in FIG. 1;

FIG. 3 is a side, cross sectional view of the components of the system shown in FIG. 1; and

FIG. 4 is a side, cross sectional view of the components of the system shown in FIG. 1.

Like reference numerals indicate similar parts throughout the figures.

### DETAILED DESCRIPTION

The exemplary embodiments of a surgical system are discussed in terms of medical devices, and more particularly to a surgical system for imaging tissue within the anatomy of a patient, such as, for example, esophageal tissue, a method for imaging an internal cavity of a patient and a kit for an endoscope. The disclosed surgical system provides, among other benefits, an enlarged imaging window for an optical probe of an endoscope to which an endoscope cap of the present disclosure is attached. It should be understood that the present disclosure is applicable to any imaging system that includes an optical probe that is inserted into an internal cavity of a patient to obtain images of the tissue surrounding the probe and/or objects present in the patient's anatomy.

In one embodiment, the surgical system includes an endoscope cap that fits on an endoscope such that the endoscope is slidably disposed within a passageway of the endoscope cap. The endoscope cap may or may not have a balloon on it. It is envisioned that the endoscope cap may be configured to slip on to the endoscope. A center piece is disposed in a passage of the endoscope cap. The center piece includes a channel that engages a channel of the endoscope. In some embodiments, at least one of the channel in the center piece and the channel in the endoscope is about 2.8 mm. An optical probe is positioned in the channel in the center piece and the channel in the endoscope. An inflation lumen is placed on the endoscope cap together with “lay flat” tubing, which is attached to the endoscope cap and runs along the endoscope.

In operation and use, the endoscope cap is fit onto one end of the endoscope. The endoscope is inserted into the anatomy of a patient, such as, for example, the esophagus of the patient. In some embodiments, the endoscope and/or endoscope cap are configured for use in other portions of the patient's anatomy to image the same, such as, for example, the patient's liver or other organs. The balloon is inflated to fix the position of the endoscope relative to the patient's anatomy. The optical probe is then loaded into and passed through the channel in the endoscope and the channel in the center piece. When the optical probe is loaded, a distal end of the endoscope engages a distal end of the endoscope cap. After the optical probe is loaded, the endoscope is translated axially within the endoscope cap such that the distal end of the endoscope is spaced apart from the distal end of the endoscope cap to define an imaging window. That is, the imaging window is a portion of the endoscope cap that is spaced apart from the endoscope. The optical probe then scans the patient's anatomy through the endoscope cap to capture an image of the patient's anatomy. After the optical probe scans the patient's anatomy, the endoscope is translated axially relative to the endoscope cap such that the distal end of the endoscope engages the distal end of the endoscope cap. The balloon is then deflated. After the balloon is deflated, at least one of the endoscope and endoscope cap are removed from the patient's anatomy.

The present disclosure may be understood more readily by reference to the following detailed description of the disclosure taken in connection with the accompanying drawing figures, which form a part of this disclosure. It is to be understood that this disclosure is not limited to the specific systems, devices, kits, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

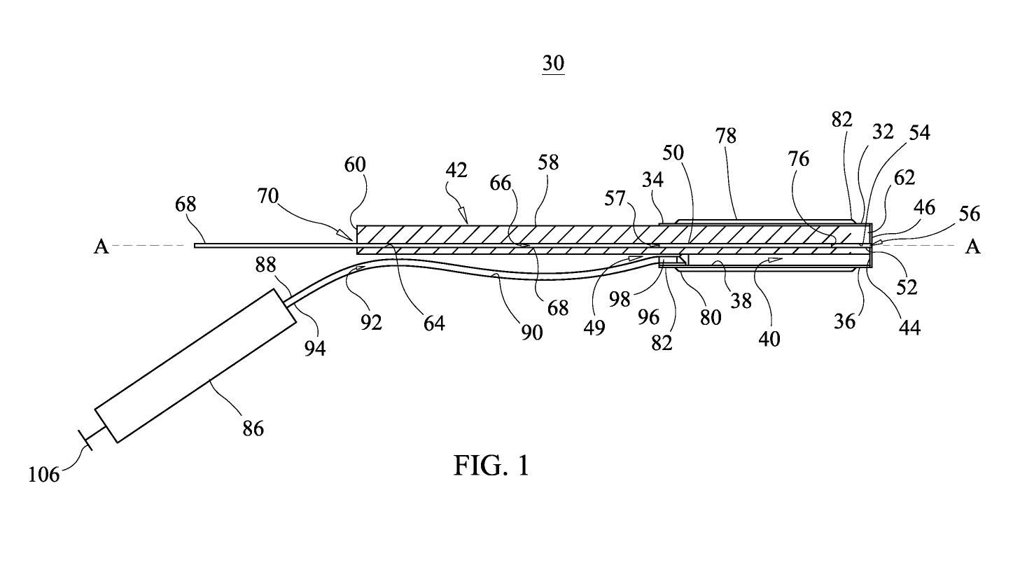

The following discussion includes a description of surgical system including an endoscope support device, such as, for example, an endoscope cap and related methods of employing the system in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there are illustrated components of a surgical system 30, in accordance with the principles of the present disclosure.

System 30 includes an elongated sleeve 32 extending along a longitudinal axis A between a first end 34 and a second end 36. Sleeve 32 has a length defined by the distance between end 34 and end 36. Sleeve 32 has a cylindrical cross sectional configuration and includes an inner surface 38 defining a cylindrical passageway 40 configured for disposal of an instrument, such as, for example, an endoscope 42, as will be described. An end surface 44 of end 36 is spaced from external surroundings by a front plate, such as, for example, a wall 46 that defines surface 44. That is, wall 46 extends perpendicular to axis A and prevents any material, such as, for example, gas, fluid or solid material from entering passageway 40 through end 36. End 34 includes a circular aperture 49 that is in communication with passageway 40 configured for insertion of endoscope 42 into passageway 40. Aperture 49 extends along a longitudinal axis B that is offset from axis A.

In some embodiments, sleeve 32 comprises a high light transmission material and/or a low light scattering material. In some embodiments, sleeve 32, including wall 46, is formed from a rigid material. In some embodiments, sleeve 32 is formed from a rigid material and wall 46 is formed from a deformable and/or elastic material. In some embodiments, sleeve 32, passageway 40 and/or aperture 49 may have various cross section configurations, such as, for example, oval, oblong, triangular, rectangular, square, polygonal, irregular, uniform, non-uniform, variable, tubular and/or tapered. In some embodiments, aperture 49 is coaxial with axis A.

An elongated cylindrical center piece 48 is disposed within passageway 40. Piece 48 is coaxial with axis A. End 52 engages surface 44 such that end 50 is spaced apart from surface 38 both before and after insertion of endoscope 42 into passageway 40, as will be described. Piece 48 has a length defined by the distance between a first end 50 and a second end 52. The length of piece 48 is less that of sleeve 32 such that end 50 is recessed relative to end 34. That is, because the length of piece 48 is less than the length of sleeve 32 and end 52 is fixed to surface 44, end 50 is positioned within passageway 40 when piece 48 engages sleeve 32. In some embodiments, the length of piece 48 is equal or greater than the length of sleeve 32. In some embodiments, piece 48 may have various cross section configurations, such as, for example, oval, oblong, triangular, rectangular, square, polygonal, irregular, uniform, non-uniform, variable, tubular and/or tapered. In some embodiments, end 52 can be variously engaged with surface 44, such as, for example, monolithic, integral connection, frictional engagement, threaded engagement, mutual grooves, screws, adhesive, nails, barbs and/or raised element. In some embodiments, piece 48 comprises a high light transmission material and/or a low light scattering material.

Piece 48 includes an inner surface 54 defining a cylindrical lumen 56 configured for slidable disposal of an optical probe 68 of endoscope 42, as will be described. Lumen 56 extends between end 50 and end 52 and has a length that is equal to the length of piece 48. In one embodiment, at end 52, lumen 56 terminates at an end wall that engages surface 44. In one embodiment, at end 52, lumen 56 terminates at surface 44. That is, surface 44 may act as a barrier that prevents material, such as, for example, gas, liquid or solid material from entering lumen 56 through end 52. Lumen 56 includes a circular opening 57 adjacent end 50. In some embodiments, lumen 56 and/or opening 57 may have various cross section configurations, such as, for example, oval, oblong, triangular, rectangular, square, polygonal, irregular, uniform, non-uniform, variable, tubular and/or tapered.

Endoscope 42 includes a cylindrical shaft 58 extending along axis B between a first end surface 60 and a second end surface 62. That is, shaft 58 is coaxial with axis B and is offset from axis A. Shaft 58 includes an inner surface 64 defining a cylindrical lumen 66 configured for slidable disposal of optical probe 68. Lumen 66 includes a first circular opening 70 adjacent surface 60 and a second circular opening 72 adjacent surface 62. Shaft 58 is positioned relative to sleeve 32 and piece 48 such that opening 57 abuts opening 72 and lumen 66 is continuous with lumen 56. That is, shaft 58 is positioned relative to sleeve 32 and piece 48 such that lumen 56 and lumen 66 form a continuous lumen that is uninterrupted by any openings between opening 70 and end 52. In some embodiments, shaft 58 comprises a rigid material. In some embodiments, shaft 58 comprises a flexible material. In some embodiments, shaft 58, opening 70 and/or opening 72 may have various cross section configurations, such as, for example, oval, oblong, triangular, rectangular, square, polygonal, irregular, uniform, non-uniform, variable, tubular and/or tapered.

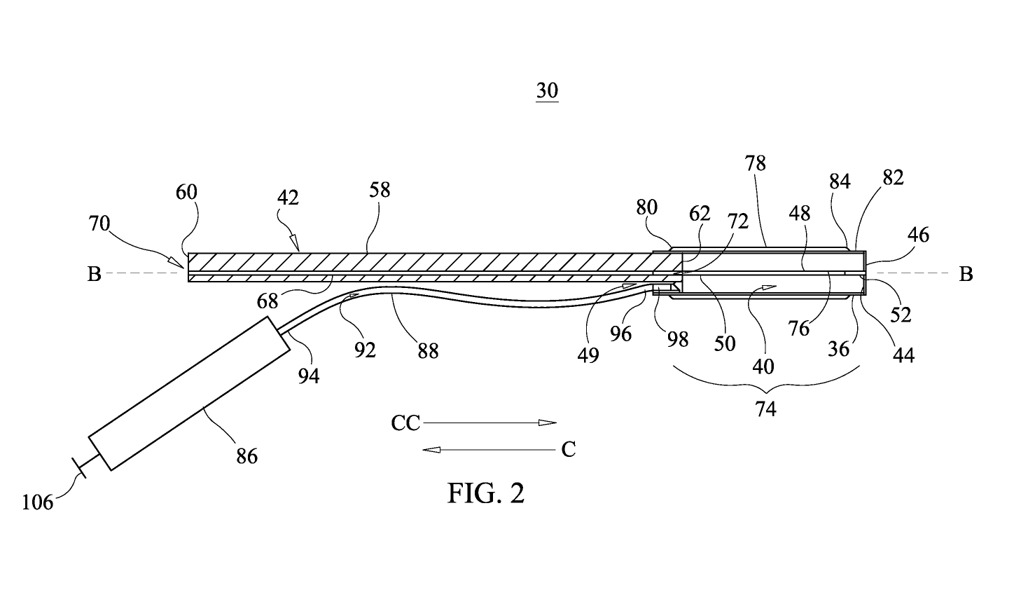

Lumen 66 has a maximum width or diameter that is greater than a maximum width or diameter of piece 48 such that piece 48 may be slidably disposed within lumen 66 as endoscope 42 moves between a first position and a second position. When endoscope 42 is in the first position, piece 48 is positioned within lumen 66 and surface 62 abuts surface 44, as shown in FIG. 1. Endoscope 42 moves from the first position to the second position by axially translating endoscope 42 within passageway 40 in the direction shown by arrow C such that opening 57 abuts opening 72, lumen 66 is continuous with lumen 56 and surface 62 is spaced apart from surface 44, as shown in FIG. 2. Endoscope 42 is moved from the second position to the first position by translating endoscope 42 within passageway 40 in the direction shown by arrow CC until piece 48 is positioned within lumen 66 and surface 62 abuts surface 44, as shown in FIG. 1.

The distance between surface 44 and surface 62 when endoscope is in the second position, or is transitioning between the first and second positions, defines an imaging window 74 of system 30. Sleeve 32 and/or piece 48 may be made from a high light transmission material and/or a low light scattering material, as discussed above, such that probe 68 may emit light and/or capture images through the portions of sleeve 32 and piece 48 that define window 74. Advancing or retracting probe 68 in window 74 allows probe 68 to take images along the entire length of window 74. Since probe 68 will take images through the walls of sleeve 32 and piece 48, the high light transmission and low light scattering material(s) used to form sleeve 32 and piece 48 assure that images taken by scope 68 through window 74 are not distorted. In some embodiments, the length of window 74 is about 2 cm to about 10 cm. In some embodiments, the length of window 74 is about 4 cm to about 8 cm. In some embodiments, the length of window 74 is about 6 cm.

Probe 68 is slidably disposed in lumen 56 and lumen 66 such that probe 68 may be translated axially within lumen 56 and lumen 66 in the direction shown by arrow C and the direction shown by arrow CC. Probe 68 has a length that is sufficient to allow a tip 76 of probe 68 to engage surface 44 when endoscope 42 is in either the first position or the second position. Probe 68 may be inserted into lumen 66 and/or lumen 56 when endoscope 42 is in the first position, shown in FIG. 1, such that tip 76 is positioned within lumen 56 while piece 48 is positioned within lumen 56 and tip 76 is spaced apart from surface 44. When endoscope 42 is moved from the first position to the second position, probe 68 may be positioned within lumen 56. Because opening 57 abuts opening 72 when endoscope 42 is in the second position, lumen 66 is continuous with lumen 56. Tip 76 may therefore be axially translated in the direction shown by arrow CC until tip 76 engages surface 44. In embodiments where sleeve 32 and/or surface 44 are made from a deformable or elastic material, probe 68 may be axially translated in the direction shown by arrow CC such that tip 76 causes surface 44 to bulge in a direction that faces away from surface 44, as shown in FIG. 2. That is, tip 76 may be selectively positioned anywhere along the length of lumen 56, depending upon, for example, position of the tissue in the anatomy of the patient that is being imaged, to optimize the position of probe 68 such that probe 68 can emit light or image tissue through window 74. Due to the configuration of system 30, once sleeve 32 is centered in an internal cavity of a patient, such as, for example, the esophagus of the patient, piece 48 assures that probe 68 will be in the center of the internal cavity, so as to allow for proper imaging of tissue that defines the internal cavity and/or objects present in the internal cavity.

In some embodiments, system 30 includes an inflatable balloon 78 that includes a first end 80 attached to an exterior surface 82 of sleeve 32 at a first point and a second end 84 attached to surface 82 at a second point that is spaced apart from the first point. Balloon 78 extends circumferentially about sleeve 32. Balloon 78 is configured to engage tissue in the anatomy of the patient such that endoscope can be selectively positioned relative to the patients anatomy, depending upon the portion of the patient's anatomy to be imaged. In some embodiments, balloon 78 comprises a compliant material, such as, for example, polyurethane, pellethane, polyethylene, silicone, cronoprene or non-compliant material such as Nylon. In some embodiments, balloon 78 comprises a high light transmission material and/or a low light scattering material. In some embodiments, balloon 78 has a uniform thickness. In some embodiments, an exterior surface of balloon 78 includes surface configurations to enhance fixation, such as, for example, rough, arcuate, undulating, porous, semi-porous, dimpled and/or textured, according to the requirements of a particular application. In some embodiments, balloon 78 is attached to sleeve 32 by heat seal, adhesive, or plastic welding in a manner that does not distort images taken through window 74 and/or balloon 78.

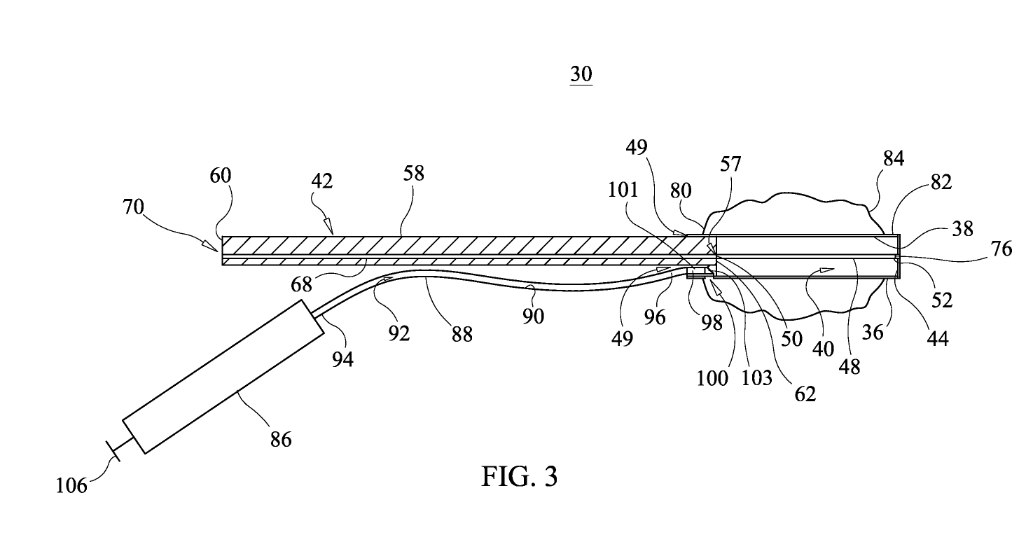

In some embodiments, end 84 is spaced apart axially from wall 46 such that at least a portion of window 74 does not have balloon 78 positioned about the circumference of window 74, as shown in FIG. 3. The portion of window 74 that does not have balloon 78 positioned about its circumference can be used to compare images taken by probe 68 through sleeve 32 and/or piece 48, with and without balloon 78, in order to make any corrections that may need to be calculated between the two images. In some embodiments, balloon 78 comprises a high light transmission material and/or a low light scattering material such that end 84 may engage wall 46 and the entire length of window 74 is covered by balloon 78.

System 30 includes an inflation source 86, such as, for example, a syringe or pump that is connected to an inflation tube 88. Tube 88 includes an inner surface 90 defining a channel 92. A first end 94 of tube 88 engages source 86 and a second end 96 of tube 88 engages a port 98 positioned within passageway 40. Port 98 includes a first portion 101 that extends parallel to axis A and a second portion 103 that extends perpendicular to axis A. Port 98 is bent between portion 101 and portion 103. An inner surface of port 98 defines a conduit that is in communication with channel 92. Port 98 is fixed relative to sleeve 32 such that port 98 does not move as endoscope 42 is moved between the first and second positions. In some embodiments, port 98 is integrally formed with sleeve 32. In some embodiments, tube 88 comprises lay flat tubing. Portion 101 includes a first opening that is in communication with the conduit of port 98 and portion 103 includes a second opening that is in communication with the conduit of port 98. The first opening of port 98 is spaced apart from passageway 40 and the second opening of port 98 opens directly into balloon such that the conduit of port 98 is not in communication with passageway 40.

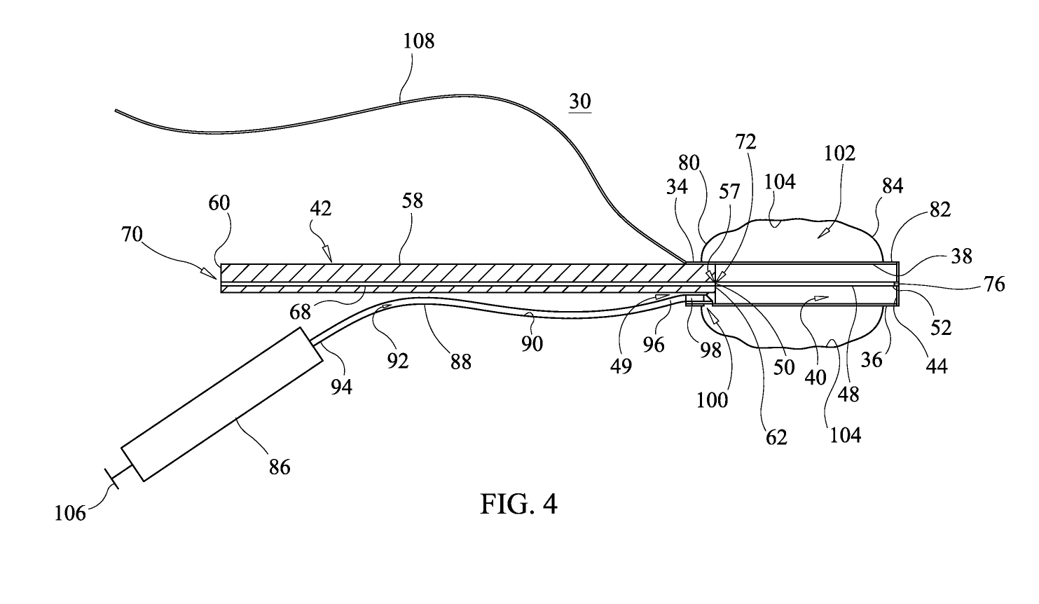

End 94 includes a first opening that is in communication with source 86 and end 96 includes a second opening that is in communication with the second opening of port 98. Sleeve 32 includes a circular opening 100 extending perpendicular to axis A through surfaces 38, 82. Opening 100 is in communication with the second opening of port 98 such that inflation material, such as, for example, saline, water, contrast fluid or compressed air contained in source 86 may be moved from source 86 through tube 88 and opening 100 and into an internal chamber 102 defined by an inner surface 104 of balloon 78. This allows balloon 78 to move from an unexpanded or collapsed configuration, shown in FIGS. 1 and 2, to an expanded configuration, shown in FIGS. 3 and 4. That is, when balloon 78 is in the unexpanded or collapsed configuration shown in FIGS. 1 and 2, inflation material from source 86 is moved into channel 92 by pressing on a plunger 106 of source 86. The inflation material then moves from channel 92 through port 98 and out of opening 100 into chamber 102. As the inflation material fills chamber 102, balloon moves to the expanded configuration shown in FIGS. 3 and 4. In some embodiments, opening 100 may have various cross section configurations, such as, for example, oval, oblong, triangular, rectangular, square, polygonal, irregular, uniform, non-uniform, variable, tubular and/or tapered. In some embodiments, the shapes and sizes of balloon 78 when in the expanded configuration can be selected to provide a desired result during a procedure. For example, balloon 78 may include shapes such as spheres, cylinders, non-uniform etc. and have different dimensions to make balloon 78 narrower or wider in a longitudinal direction, or extend further in a radial direction, etc.

In operation and use, system 30 is inserted into an internal cavity of a patient, such as, for example, the esophagus of the patient, with endoscope in the first position, shown in FIG. 1. Sleeve 32 is translated within the patient's esophagus until sleeve 32 is positioned adjacent tissue or an object in the patient's esophagus to be imaged. Once sleeve 32 is in the desired location, balloon 78 is moved from the collapsed orientation to the expanded orientation by injecting inflation material from source 86 through tube 88 and port 98 and into chamber 102. Balloon 78 is filled with the inflation material until sleeve 32 has a diameter sufficient to engage an outer surface of balloon 78 with tissue in the patient's anatomy in a manner that fixes sleeve 32 relative to the patient's anatomy. The diameter of sleeve 32 is configured such that balloon 78 need only be inflated a minor amount, since sleeve 32 is designed to take up a substantial part of the internal cavity of the patient, such as, for example the esophagus. That is, when used for esophageal imaging, sleeve 32 will have a diameter that closely matches the diameter of the patient's esophagus, while allowing movement of sleeve 32 within the esophagus.

Once balloon 78 is moved from the collapsed configuration to the expanded configuration to fix sleeve 32 relative to the esophagus, endoscope 42 is moved from the first position, shown in FIG. 1, to the second position, shown in FIG. 2. Moving endoscope 42 from the first position to the second position creates window 74. Probe 68 is then translated through lumens 56, 66 until tip 76 is positioned at a selected location relative to window 74. Images of the patient's tissue or objects within the patient's anatomy are then taken through window 74 using probe 68.

Once the desired images of the patient and/or objects within the patient have been taken, balloon 78 can be moved from the expanded orientation, shown in FIGS. 3 and 4, to the collapsed orientation, shown in FIGS. 1 and 2. Endoscope 42 is then moved from the second position, shown in FIG. 2, to the first position, shown in FIG. 1. System 30, including at least one of sleeve, piece 48, endoscope 42, source 86 and tube 88 may then be removed from the patient's anatomy.

Where this application has listed the steps of a method or procedure in a specific order, it may be possible, or even expedient in certain circumstances, to change the order in which some steps are performed, and it is intended that the particular steps of the method or procedure claim set forth herein below not be construed as being order-specific unless such order specificity is expressly stated in the claim.

In one embodiment, in accordance with the principles of the present disclosure, sleeve 32 can be equipped with a tether 108. Tether 108 may be fixed along any portion of sleeve. As shown in FIG. 4, tether 108 is fixed to surface 82 adjacent end 34. Tether 108 has a length that is long enough to extend out of an internal cavity of a patient, such as, for example, the patient's esophagus, and can be used in case sleeve 32 becomes dislodged during an application. In some embodiments, tether 108 comprises lay flat tubing.

In one embodiment, one of sleeve 32 and endoscope 42 include a protrusion configured to engage a notch the other of sleeve 32 and endoscope 42 when endoscope 42 is in the first position so as to prevent sleeve 32 from becoming dislodged as it is retracted out of the patient. In one embodiment, one of sleeve 32 and endoscope 42 include a protrusion configured to engage a notch the other of sleeve 32 and endoscope 42 when endoscope 42 is in the second position so as to provisionally lock endoscope 42 in the second position. This will allow a medical practitioner to image tissue and/or objects within the patient's anatomy using probe 68 without having endoscope moving from the second position to the first position during imaging.

In one embodiment, system 30 includes lay flat tubing that covers endoscope 42, at least in part, so as to protect endoscope 42 from bodily fluid and damage during use. The lay flat tubing has ultra-thin walls and is made of high quality polymers. The lay flat tubing takes up minimal space while offering strength, lubricity, chemical inertness, and biocompatibility.

In one embodiment, in accordance with the principles of the present disclosure, a kit for an endoscope is provided comprising at least one sleeve 32, at least one endoscope 42, at least one piece 48, at least one balloon 78, at least one source 86 and at least one tube 90. Tube 88 can be equipped with a relief valve to prevent over inflating of balloon 78. In one embodiment of the kit, additional tubing 80, a variety of different sized balloons 78, and different sized sleeves 32 and pieces 48 can be provided as well.

Since the components of the endoscope device will be inserted into an internal cavity of a patient, it is advantageous to provide components that are biologically acceptable materials suitable for medical applications. The biologically acceptable materials suitable for medical applications for the components of system 30 can be fabricated from and include metals, synthetic polymers, ceramics and bone material and/or their composites, depending on the particular application and/or preference of a medical practitioner. For example, the components of system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), PVC, fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, and their combinations.

Various components of system 30 may have material composites, including the above materials, to achieve various desired characteristics such as strength, rigidity, elasticity, compliance, mechanical performance and durability. The components of system 30, individually or collectively, may also be fabricated from a heterogeneous material such as a combination of two or more of the above-described materials. The components of system 30 may be monolithically formed, integrally connected or include fastening elements and/or instruments, as described herein.

While the preferred embodiments of the devices and methods have been described in reference to the environment in which they were developed, they are merely illustrative of the principles of the present disclosure. Modification or combinations of the above-described devices, systems, kits and methods, and variations thereof that are obvious to those of skill in the art are intended to be within the scope of the present disclosure.

What is claimed is:

1. An endoscope support device comprising:
a sleeve extending along a longitudinal axis between a first end and a second end, the sleeve comprising an inner surface defining a passageway configured for disposal of an endoscope, an end surface of the distal end being spaced from external surroundings by a wall that defines the end surface; and
a center piece disposed within the passageway, the center piece comprising a first end that engages the end wall and an inner surface defining a lumen configured for disposal of an optical probe, the center piece being positioned within the passageway such that the lumen is aligned with a lumen of the endoscope and the probe extends through the lumens.

2. An endoscope support device as recited in claim 1, further comprising an inflatable balloon comprising a first end attached to an exterior surface of the sleeve at a first point and a second end attached to the exterior surface at a second point, the balloon being attached to an inflating device that directs fluid from the inflating device to an interior of the balloon to expand the balloon about the sleeve.

3. An endoscope support device as recited in claim 2, wherein:
the sleeve comprises an opening extending through the inner surface of the sleeve and the exterior surface of the sleeve, the opening being in communication with the interior of the balloon; and
the inflating device comprises an inflation tube comprising an inner surface defining a passage, a first end of the inflation tube engaging the sleeve such that the passage is in communication with the opening.

4. An endoscope support device as recited in claim 2, wherein the first and second points are axially spaced apart from the wall.

5. An endoscope support device as recited in claim 2, wherein the balloon extends circumferentially about the exterior surface of the sleeve.

6. An endoscope support device as recited in claim 1, wherein at least one of the sleeve and the center piece is constructed of high light transmission material.

7. An endoscope support device as recited in claim 1, wherein at least one of the sleeve and the center piece is constructed of low light scattering material.

8. An endoscope support device as recited in claim 1, wherein the sleeve and the center piece are coaxial.

9. An endoscope support device as recited in claim 1, wherein the wall comprises an elastic material.

10. A method for imaging a patient comprising;
providing the endoscope support device of claim 2;
positioning an end portion of an endoscope into the passageway so that the center piece is coaxial with an optical probe of the endoscope;
positioning the endoscope support device into anatomy of a patient;
advancing the optical probe through a probe assembly lumen of the endoscope and into the lumen of the center piece;
inflating the balloon so that the balloon contacts at least a portion of a wall defining an internal cavity of a patient so as to hold and/or center the device within the internal cavity; and
activating the optical probe to obtain and record images of at least a portion of a tissue defining the internal cavity.

11. A method for imaging a patient as recited in claim 10, further comprising sliding the endoscope within the passageway between a first position in which an end face of the endoscope is spaced apart from the end surface and a second position in which the end face engages the end surface.

12. A method for imaging a patient as recited in claim 11, wherein the optical probe is activated when the endoscope is in the first position.

13. A kit for an endoscope comprising;
at least one support device of claim 2;
tubing for supplying air from the inflating device to the interior of the balloon;
additional replacement tubing attachable to the air supply device; and
additional balloons attachable to the sleeve.

14. An endoscope support device comprising:
a sleeve extending along a longitudinal axis between a first end and a second end, the sleeve comprising an inner surface defining a passageway configured for disposal of an endoscope, an end surface of the distal end being spaced from external surroundings by a wall that defines the end surface;
a center piece disposed within the passageway, the center piece comprising a first end that engages the end wall and an inner surface defining a lumen configured for disposal of an optical probe, the center piece being positioned within the passageway such that the lumen is aligned with a lumen of the endoscope and the probe extends through the lumens;
an inflatable balloon comprising a first end attached to an exterior surface of the sleeve at a first point and a second end attached to the exterior surface at a second point; and
an inflating device having a first end positioned in the passageway, the inflating device comprising an inner surface that defines a channel that is communication with an interior of the balloon such that the channel directs fluid from the inflating device to the interior of the balloon to expand the balloon about the sleeve.

15. An endoscope support device as recited in claim 14, wherein the center piece is coaxial with the sleeve and the inflating device defines an axis that is offset from the longitudinal axis.

16. An endoscope support device as recited in claim 14, wherein the sleeve comprises an opening extending transverse to the longitudinal axis through the inner surface of the sleeve and an exterior surface of the sleeve, the channel being in communication with the opening.

17. A surgical system comprising:
an elongated sleeve extending along a longitudinal axis between a first end and an opposite second end, the sleeve comprising an inner surface defining a passageway, an end surface of the second end being spaced from external surroundings by a wall that defines the end surface;
an elongated center piece disposed within the passageway, the center piece comprising a first end that engages the end wall and an inner surface defining a lumen; and
an endoscope disposed within the passageway adjacent the first end of the sleeve, the endoscope comprising an inner surface defining an optical probe lumen that is continuous with the lumen of the center piece, the endoscope comprises an optical probe positioned in the lumen of the center piece and the optical probe lumen,
wherein at least one of the sleeve and the center piece is constructed of high light transmission and low light scattering material.

18. A surgical system as recited in claim 17, further comprising an inflatable balloon comprising a first end attached to an exterior surface of the sleeve at a first point and a second end attached to the exterior surface at a second point, the balloon being attached to an inflating device that directs fluid from the inflating device to an interior of the balloon to expand the balloon about the sleeve.

19. A surgical system as recited in claim 17, wherein the endoscope is slidably disposed within the passageway.

20. A surgical system as recited in claim 17, wherein the center piece is coaxial with the sleeve and the endoscope defines an axis that is offset from the longitudinal axis.
